(19) United States
(12) Patent Application Publication
(10) Pub. No.: US 2023/0001222 A1
(43) Pub. Date: Jan. 5, 2023

(54) Flexible Photobiomodulation and Pulsed Electromagnetic Field Therapy Device

(71) Applicant: Jason Henry Sousa, Agoura Hills, CA (US)

(72) Inventor: Jason Henry Sousa, Agoura Hills, CA (US)

(21) Appl. No.: 17/521,926

(22) Filed: Nov. 9, 2021

Related U.S. Application Data
(60) Provisional application No. 63/216,594, filed on Jun. 30, 2021.

(51) Int. Cl. A61N 2/00; A61N 5/06

(52) U.S. Cl. CPC A61N 2/002; A61N 2/004; A61N 5/062; A61N 2005/0645; A61N 2005/0651; A61N 2005/0659; A61N 2005/0663

(57) ABSTRACT

The present disclosure provides a combined Pulsed Electromagnetic Field (PEMF) therapy and Photobiomodulation (PBM) therapeutic device in the form of a flexible wearable that can be secured to a user's body part to apply targeted radiation to a desired area. The frequencies and pulse patterns of both types of radiation are controlled by a single controller of the device which can switch between a number of pre-set sequences and patterns. The device is wirelessly enabled to allow control via an external user device.

### CROSS REFERENCES TO RELATED APPLICATIONS

The present application claims the benefit and priority of U.S. provisional application No. 63/216,594, filed 30 Jun. 2021.

### FIELD OF INVENTION

This invention relates generally to therapeutic devices, and more specifically to a pulsed electromagnetic field, PEMF, and phototherapy device having pre-set frequencies and programs for applying therapeutic stimulation to a target area of a patient's body.

### BACKGROUND

Pulsed Electromagnetic Field (PEMF) therapy is a safe and non-invasive way to reduce pain and inflammation and to promote healing of bones and other body tissues. It applies non-damaging electromagnetic frequencies to enhance overall health and wellness.

Every cell in the body has a negative charge on the cell wall. For nerve cells, this is approximately −60 mV; other cells may vary in the net negative charge. To maintain healthy levels of this negative charge, potassium and magnesium should be contained inside the cells, while calcium and sodium ideally remain outside the cells. The human diet is often insufficient to maintain a proper balance of these chemicals in all cells, especially after injury and trauma—PEMF is used to correct for this to ensure the electrical health of a patient's cells. PEMF can be used to supplement and enhance currently existing healthcare modalities, improving adenosine triphosphate [ATP] production, increasing oxygenation, enhancing circulation, promoting hydration, facilitating detoxification, and gaining a better overall absorption of nutrients.

Photobiomodulation (PBM Therapy) is the application of red and near infra-red light over injuries, joints, or lesions to improve wound and soft tissue healing, reduce inflammation and give relief for both acute and chronic pain.

The red and near infrared light (600 nm-1000 nm) commonly used in PBM can be produced by laser or high intensity LEDs. When the correct intensity and treatment times are used, red and near infrared light reduces oxidative stress and increases ATP. This improves cell metabolism and reduce inflammation by inducing nitric oxide production, blood flow and also movement of growth factor and increase cellular respiration. These effects can be enhanced by pulsed emissions.

Despite both PEMF and PBM therapies being non-invasive and effective in supporting the body's healing processes and overall health (both have been shown to shown to heal muscle and ligament damage and reduce delayed onset muscle soreness), there are currently no devices available on the market for applying these two synergistic therapies in tandem.

Existing PEMF and PBM solutions apply each therapy in isolation, and furthermore they are often contained in cumbersome hard plastic or metal enclosures that do not contour to the body and required a user to stay in place while either therapy was applied. It is within this context that the present invention is provided.

### SUMMARY

The present disclosure provides a combined Pulsed Electromagnetic Field (PEMF) therapy and Photobiomodulation (PBM) therapeutic device in the form of a flexible wearable that can be secured to a user's body part to apply targeted radiation to a desired area. The frequencies and pulse patterns of both types of radiation are controlled by a single controller of the device which can switch between a number of pre-set sequences and patterns. The device is wirelessly enabled to allow control via an external user device.

Thus, according to one aspect of the present disclosure there is provided a wearable device for applying phototherapy and pulsed electromagnetic field, PEMF, therapy to a user, the device comprising: a flexible substrate having a first surface and one or more fastening loops configured to secure the substrate to a body part with the first surface in contact with the skin of a body part; a power source; and one or more controls.

The device further comprises one or more electromagnetic coils disposed on the first surface; one or more first diodes configured to emit a first frequency of light, the one or more first diodes being disposed on the first surface; one or more second diodes configured to emit a second frequency of light, the one or more second diodes being disposed on the first surface; a wireless communications module; and a control module.

The control module is configured to, in response to instructions received via the one or more controls or via the wireless communications module, cause the electromagnetic coils, the first diodes, and the second diodes, to each emit pulses of radiation at a respective frequency according to one of a plurality of pre-set frequency sequences stored in the control module memory.

In some embodiments, the first frequency is in the red light wavelength range of 620 nm to 750 nm.

In some embodiments, the second frequency is in the near infra-red range of 800 nm to 2500 nm.

In some embodiments, the fastening loops comprise securing means for locking the device in place in contact with the body part. The securing means may comprise one or more hook and loop mechanisms.

In some embodiments, a plurality of different frequency sequence programs are stored within the memory of the control module and actuation of the one or more controls cause the control module to cycle between programs.

The frequency sequence programs may comprise a Program A which causes pulsed radiation at 73 hz, 587 hz, and 1174 hz in sequence, a Program B which causes pulsed radiation at 293 hz, 587 hz, 1174 hz, and 4698 hz in sequence, and a Program C which causes pulsed radiation at 73 hz, 146 hz, 293 hz, 587 hz, 1174 hz, 2349 hz, and 4698 hz in sequence.

In some embodiments, the control module causes the first diodes and second diodes to emit radiation with the same frequency.

In some embodiments, the wireless communications module comprises Bluetooth capabilities.

In some embodiments, the controller is configured to interact with a dedicated application software installed on a user device via the wireless communications module.

In some embodiments, the control module is configured to start a timer upon the device being switched on and to automatically power down the device when the timer reaches a pre-set time limit.

In some embodiments, the power source is a rechargeable battery and the device further comprises a charging input and circuit.

In some embodiments, the substrate is in the form of a flat pad having a square shape.

In some embodiments, the substrate is in the form of a shoulder pad having rounded edges.

In some embodiments, the substrate is made of a fabric material.

### BRIEF DESCRIPTION OF THE DRAWINGS

Various embodiments of the invention are disclosed in the following detailed description and accompanying drawings.

FIG. 1 illustrates a first isometric view of an example configuration of the device of the present disclosure.

FIG. 2 illustrates a second isometric view of an example configuration of the device of the present disclosure.

FIG. 3 illustrates a frontal view of an example configuration of the device of the present disclosure, showing the inner surface to be in contact with a user.

FIG. 4 illustrates a rear view of an example configuration of the device of the present disclosure, showing the outer surface and protective housing for the circuitry.

FIG. 5 illustrates a first side view of an example configuration of the device of the present disclosure, shown along the length.

FIG. 6 illustrates a second side view of an example configuration of the device of the present disclosure, shown along the length.

FIG. 7 illustrates a third side view of an example configuration of the device of the present disclosure, shown along the width.

FIG. 8 illustrates a fourth side view of an example configuration of the device of the present disclosure, shown along the width.

FIG. 9 illustrates a circuit diagram of an example power supply circuit for the device of the present disclosure.

FIG. 10 illustrates a circuit diagram of an example control interface circuit for the device of the present disclosure.

FIG. 11 illustrates a circuit diagram of an example power regulation circuit for the device of the present disclosure.

FIG. 12 illustrates a circuit diagram of an example process controller circuit and chip for the device of the present disclosure.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

The terminology used herein is for the purpose of describing particular embodiments only and is not intended to be limiting of the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

As mentioned above, the present disclosure provides a therapeutic device in the form of a flexible pad which can be affixed to a target body part of a wearer and which has both PEMF and PBM therapy elements disposed on a surface that will be in contact with the target body area of the user.

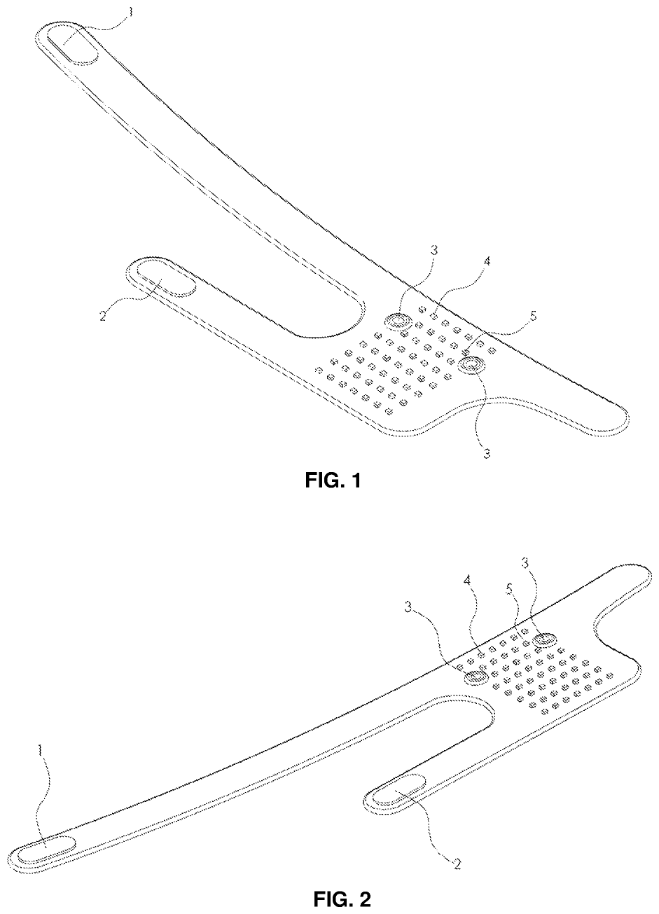

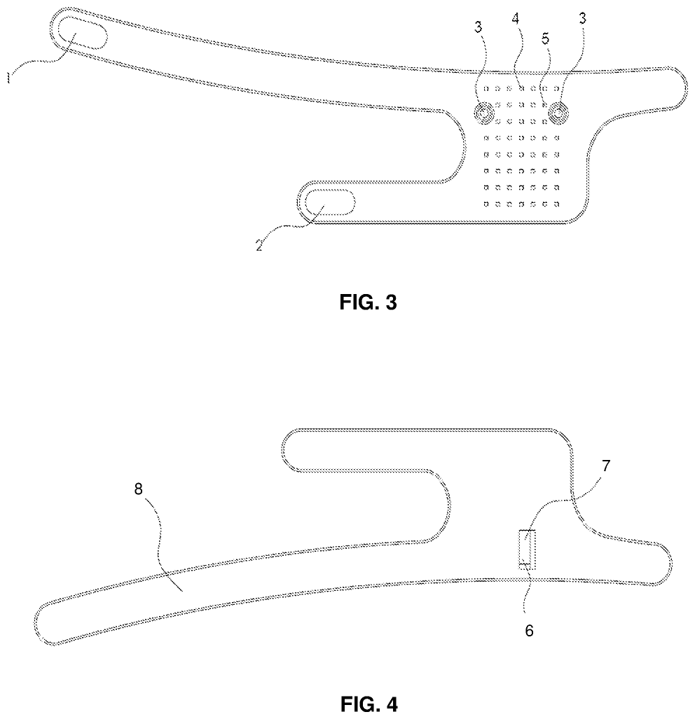

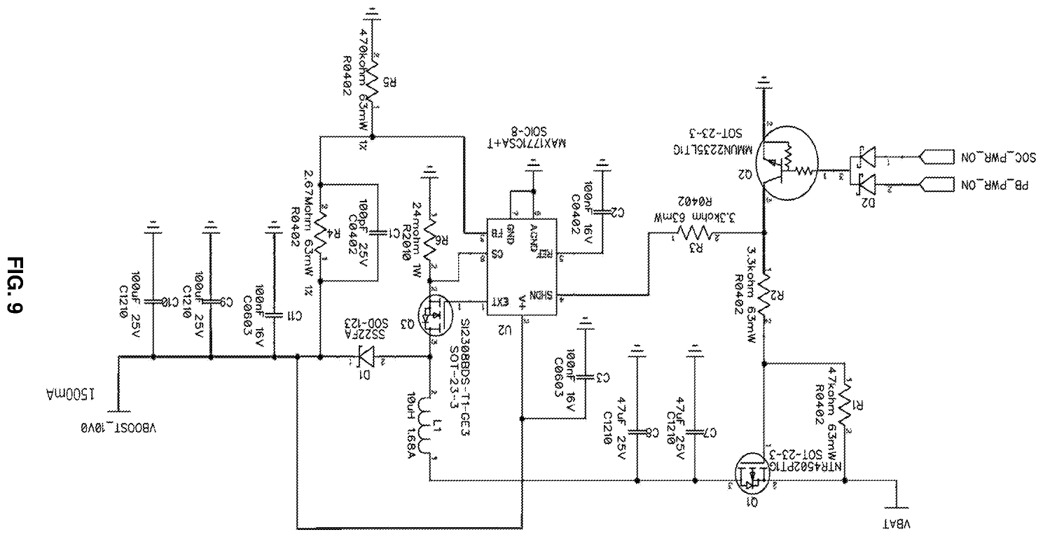

One example configuration of the disclosed device is shown in detail with reference to FIGS. 1-8.

Referring to FIGS. 1-3, the inner surface of the device is shown. The device is in the form of a fabric pad with two fastening loops extending from the pad, a first longer fastening loop with an opposing connector element and a second shorter fastening loop. The longer fastening loop has a first securing means 1 for connecting to the opposing connector element and the shorter fastening loop has a second securing means 2 for connecting to any other part of the device.

This configuration allows the device to be versatile in its placement, affixing to effectively any part of the human body. In some examples the securing means are hook and loop mechanisms with opposing parts on the opposite surface of the device.

The inner surface of the pad of the device has disposed thereon two electromagnetic coils 3 configured to emit pulsed radiation. The same surface also has disposed thereon a plurality of first diodes 4 configured to emit a first frequency of photobiomodulation light, for example red light, and a second plurality of diodes 5 configured to emit photobiomodulation light, for example Near Infrared light. In the present example the diodes are arranged in a grid and interspersed with each other but other configurations are possible.

Referring to FIG. 4 the opposing surface of the device is shown. The circuitry and control elements required to cause the coils 3 and diodes 4 and 5 to emit pulsed radiation according to one or more pre-set sequences are disposed within a protective housing 6 comprising an integrated circuit board 7 for both PEMF and PBM control, power source, wireless communications module, and microcontroller.

The rear surface of the device shown in FIG. 4 may be composed of the same piece of material 8 as the inner surface of may comprise a protective fabric backing 8.

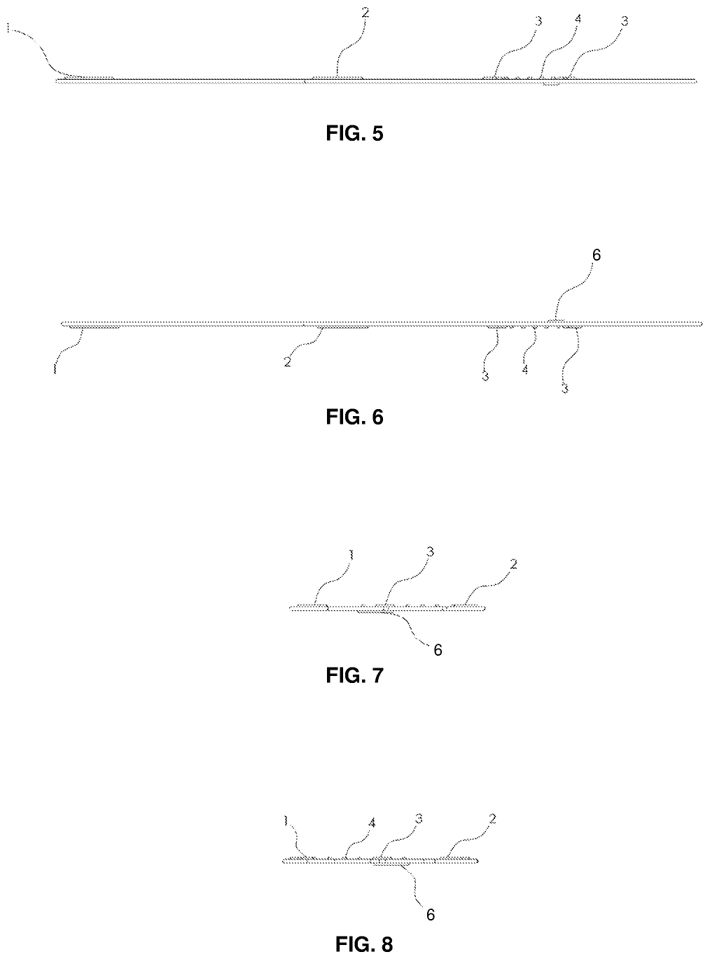

FIGS. 5-8 show various side views of the example configuration of the device. As can be seen, the flexible substrate forming the pad and loops is kept thin and the therapeutic and circuitry components only extend a minimal distance from the flat surface to ensure user comfort.

FIGS. 9-12 show various example functional circuits suitable for powering and operating the device of the present disclosure.

In particular, FIG. 9 illustrates a circuit diagram of an example power supply circuit for the device of the present disclosure which links a lithium polymer battery power source and steps it up to 10V for powering the radiating elements including the coils and the diodes, as well as a connecting circuit leading to the microcontroller and wireless communications module circuits.

Thus, in the example circuit shown, the values chosen for the components are chosen based on the following example parameters: a power source of a 3.7V to 4.5V Li-Ion Battery; 1800 mAH, 2×PEMF coil loads (estimated 250 mA each), the diode grid/array (in the present example comprising 23 branches of 2 LEDs per branch) of 46×20 mA LEDs.

The output power rating of the example device is thus approximately: 2*10V*0.25 A+23*6V*0.02 A+2.2V*500 mA=10 W.

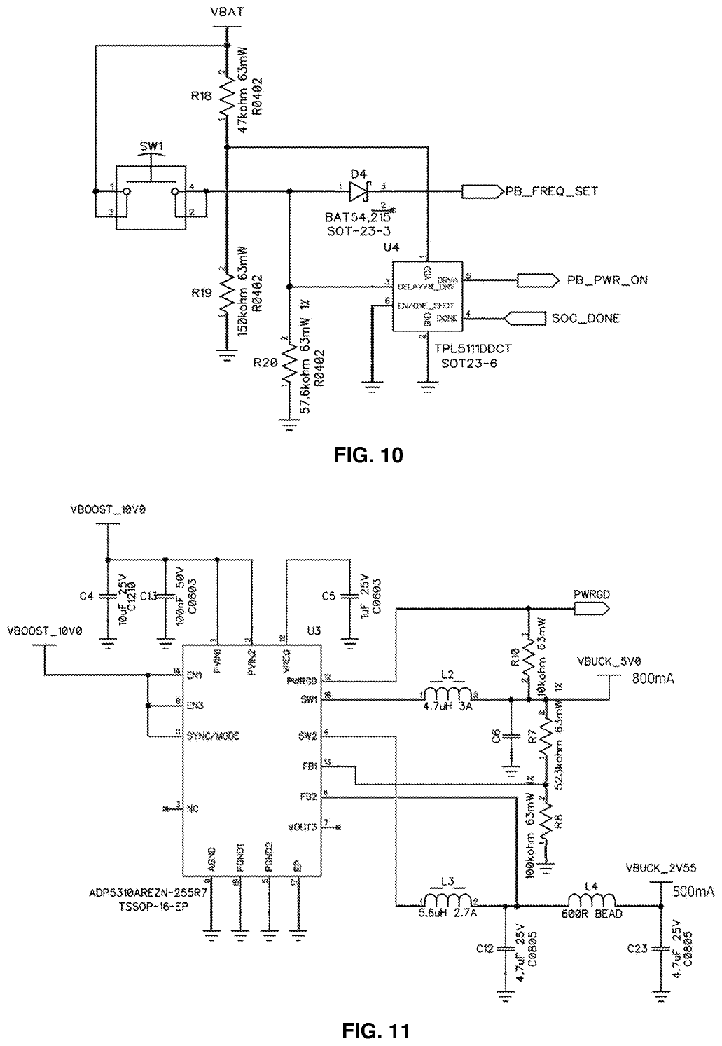

FIG. 10 illustrates a circuit diagram of an example control interface circuit for the device of the present disclosure in the from of a one shot/momentary push button for both powering on the device and changing the frequency setting.

The pushbutton allows the user to power-ON the device, activating the 2×PEMF coils at a first frequency of XX Hz pulsing and turning on all the LEDs at a second frequency of YY Hz pulsing. X and Y can be the same or different.

A subsequent actuation of the button will set the frequency of the diodes and the coils according to one of a plurality of pre-set the sequences stored in the memory of the microcontroller or built into the circuit itself. Subsequent pushes will cause the device to cycle through different pre-set frequency sequences.

FIG. 11 illustrates a circuit diagram of an example multi-output power regulation circuit for the device of the present disclosure that connects the 10V booster circuit of FIG. 9 to the radiating elements and control circuitry.

Specifically, in the present example the power regulation circuit splits and steps down the 10V input from the power supply circuit to: a 5V output to power both types of LEDs; and a 2.55V output to power the microcontroller SoC (System-on-Chip).

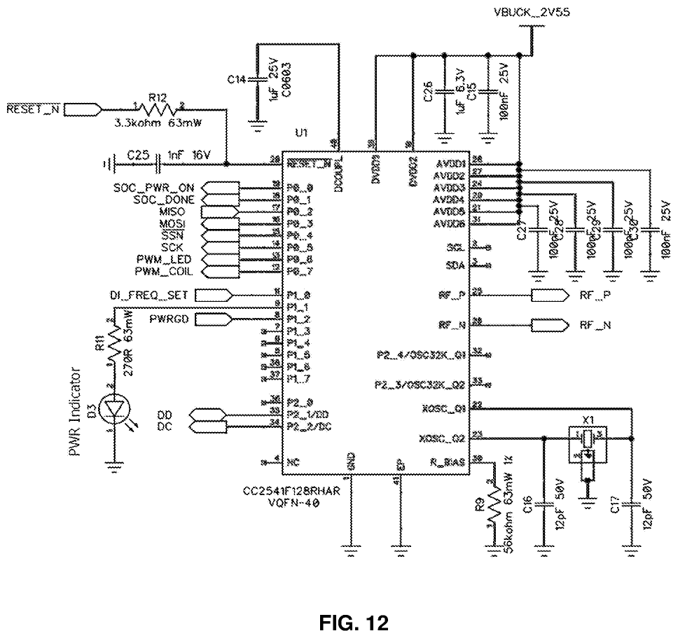

FIG. 12 illustrates a circuit diagram of an example SoC process controller circuit for the device of the present disclosure.

This circuit controls and regulates the pwm outputs which drive the PBM LEDs and PEMF coils, pulsing them according to one of a plurality of frequency sequences stored in its memory based on instructions received from either the control circuit of FIG. 10 or received externally from a user device such as a smart phone having dedicated application software for interfacing with the device installed thereon.

In order to communicate with an external user device, the circuit of FIG. 12 either has a wireless transceiver integrated with it or is coupled to a wireless communication module circuit as is known to those skilled in the art. The wireless communications module may be Bluetooth enabled but may also be capable of other wireless communications standards.

If the device is controlled by the user through Bluetooth as such, the user can both select one of a plurality of pre-set frequency pulse sequences for the device and remotely turn the device off at any time through an interface of their user device. The user may also be able to create custom frequency sequences on the application software of their user device.

An example set of frequency sequence programs stored on the device are as follows:

Program A: 73 hz+587 hz+1174 hz

Program B: 293 hz+587 hz+1174 hz+4698 hz

Program C: 73 hz+146 hz+293 hz+587 hz+1174 hz+2349 hz+4698 hz

Unless otherwise instructed the controller circuit will cause the device to automatically turn off a pre-set time after switching on, such as for example after 10 minutes of operation.

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the therapeutic device have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A wearable device for applying phototherapy and pulsed electromagnetic field, PEMF, therapy to a user, the device comprising:
a flexible substrate having a first surface and one or more fastening loops configured to secure the substrate to a body part with the first surface in contact with the skin of a body part;
a power source;
one or more controls;
one or more electromagnetic coils disposed on the first surface;
one or more first diodes configured to emit a first frequency of light, the one or more first diodes being disposed on the first surface;
one or more second diodes configured to emit a second frequency of light, the one or more second diodes being disposed on the first surface;
a wireless communications module; and
a control module, the control module being configured to, in response to instructions received via the one or more controls or via the wireless communications module, cause the electromagnetic coils, the first diodes, and the second diodes, to each emit pulses of radiation at a respective frequency according to one of a plurality of pre-set frequency sequences stored in the control module memory.

2. A wearable device according to claim 1, wherein the first frequency is in the red light wavelength range of 620 nm to 750 nm.

3. A wearable device according to claim 1, wherein the second frequency is in the near infra-red range of 800 nm to 2500 nm.

4. A wearable device according to claim 1, wherein the fastening loops comprise securing means for locking the device in place in contact with the body part.

5. A wearable device according to claim 4, wherein the securing means comprise one or more hook and loop mechanisms.

6. A wearable device according to claim 1, wherein a plurality of different frequency sequence programs are stored within the memory of the control module and actuation of the one or more controls cause the control module to cycle between programs.

7. A wearable device according to claim 6, wherein the frequency sequence programs comprise a Program A which causes pulsed radiation at 73 hz, 587 hz, and 1174 hz in sequence, a Program B which causes pulsed radiation at 293 hz, 587 hz, 1174 hz, and 4698 hz in sequence, and a Program C which causes pulsed radiation at 73 hz, 146 hz, 293 hz, 587 hz, 1174 hz, 2349 hz, and 4698 hz in sequence.

8. A wearable device according to claim 1, wherein the control module causes the first diodes and second diodes to emit radiation with the same frequency.

9. A wearable device according to claim 1, wherein the wireless communications module comprises Bluetooth capabilities.

10. A wearable device according to claim 1, wherein the controller is configured to interact with a dedicated application software installed on a user device via the wireless communications module.

11. A wearable device according to claim 1, wherein the control module is configured to start a timer upon the device being switched on and to automatically power down the device when the timer reaches a pre-set time limit.

12. A wearable device according to claim 1, wherein the power source is a rechargeable battery and the device further comprises a charging input and circuit.

13. A wearable device according to claim 1, wherein the substrate is in the form of a flat pad having a square shape.

14. A wearable device according to claim 1, wherein the substrate is in the form of a shoulder pad having rounded edges.

15. A wearable device according to claim 1, wherein the substrate is made of a fabric material.
